(19) United States
(12) Patent Application Publication
(10) Pub. No.: US 2025/0388938 A1
(43) Pub. Date: Dec. 25, 2025

(54) SEMI-INTEGRATED METHOD FOR ENZYME-ASSISTED EXTRACTION AND PURIFICATION OF P-HYDROXYCINNAMIC ACIDS

(71) Applicant: Institut des Sciences et Industries du Vivant et de l'environnement - AgroParisTech, Paris Cedex 05 (FR)

(72) Inventors: Florent Allais, Bouy (FR); Louis Mouterde, Reims (FR); Irina Ioannou, Bazancourt (FR); Valentin Reungoat, Reims (FR); Morad Chadni, Reims (FR); Amandine Flourat, La Neuvillette, Reims (FR); Hèléne Ducatel, Beauvoir (FR)

(21) Appl. No.: 18/727,462

(22) PCT Filed: Jan. 13, 2023
(86) PCT No.: PCT/EP2023/050752

§ 371 Date: Feb. 25, 2025

(30) Foreign Application Priority Data
Jan. 13, 2022 (FR) FR2200267

(51) Int. Cl. C12P 7/42; B01D 11/02; B01D 11/04; C12N 9/18

(52) U.S. Cl. CPC C12P 7/42; B01D 11/0288; B01D 11/0415; C12N 9/18; C12Y 301/01073

(57) ABSTRACT

A semi-integrated method for production of p-hydroxycinnamic acids comprises a first step of enzymatic hydrolysis and a second step of purification of the p-hydroxycinnamic acids, thus released, by liquid/liquid extraction using a membrane contactor. This method enables the purification of p-hydroxycinnamic acids from a plant biomass and the recovery thereof in the organic phase. It is conceivable to recover the p-hydroxycinnamic acids in the aqueous phase after a back-extraction step.

### CROSS REFERENCE TO RELATED APPLICATIONS

This application is a national phase entry under 35 U.S.C. § 371 of International Patent Application PCT/EP2023/050752, filed Jan. 13, 2023, designating the United States of America and published as International Patent Publication WO 2023/135259 A1 on Jul. 20, 2023, which claims the benefit under Article 8 of the Patent Cooperation Treaty to French Patent Application Serial No. FR2200267, filed Jan. 13, 2022.

### TECHNICAL FIELD

The present disclosure relates to the field of the production of p-hydroxycinnamic acids from lignocellulosic plant biomass. More particularly, the present disclosure relates to a semi-integrated method comprising a first step of enzymatic hydrolysis and a second step of purification of the p-hydroxycinnamic acids, thus released, by liquid/liquid extraction using a membrane contactor. This method enables the purification of p-hydroxycinnamic acids from a lignocellulosic plant biomass and the recovery thereof in the organic phase. It is conceivable to recover the p-hydroxycinnamic acids in the aqueous phase after a back-extraction step.

### BACKGROUND

P-hydroxycinnamic acids are molecules with a wide range of biological activities (antioxidant, anti-inflammatory, antimicrobial, etc.). They are also synthons of choice for the production of anti-UV, antioxidant and antimicrobial molecules, bisphenol A substitutes and bio-based monomers and polymers.

P-hydroxycinnamic acids can currently be obtained by chemical synthesis, biotechnology or biomass recovery.

The most widely used chemical synthesis route is the Knoevenagel-Doebner condensation between malonic acid and the corresponding aldehyde, using pyridine as a solvent and aniline as a catalyst. Developments have been made to make the synthesis greener, by substituting pyridine with ethanol and aniline with proline, but the resulting p-hydroxycinnamic acids cannot be labeled as “natural molecules.”

As far as biotechnological production is concerned, p-hydroxycinnamic acids can be produced by constructing and inserting a metabolic pathway in yeast and overexpressing the corresponding genes. The molecules obtained can be considered natural, but only the p-Coumaric acid production route is currently sufficiently productive to be industrialized.

The 3^{rd} route is to obtain these acids by extraction and purification from biomass. In this case, numerous unit operations must be carried out to obtain p-hydroxycinnamic acids with a high degree of purity, as these molecules are present in plant biomass at a level of 1 to 2% by mass.

Numerous studies have been published in the literature, but few have focused on the development of a p-hydroxycinnamic acid production chain from extraction to purification. Although high yields can be achieved after purification, production costs remain an obstacle to industrialization. There are two possible scenarios: either the acid or the derivatives thereof are present in free form (for example, in mustard bran, rapeseed cake, chicory co-products), or they are present in a form bound to plant walls (for example, in wheat bran). In the first case, it is necessary to extract the molecule and then optionally convert it into p-hydroxycinnamic acid. This conversion is usually carried out using alkaline solutions. In the second case, alkaline hydrolysis releases the acids from the biomass walls, leading to near-simultaneous extraction in the medium. In both cases, residual liquid media can be purified using a variety of technologies: liquid-liquid extraction, membrane processes, absorption, adsorption or ion exchange media. However, the use of alkaline solutions leads to a loss of naturalness in the molecules produced. Some studies have favored the use of enzymes, thus preserving the naturalness of the molecules, although it is necessary to adapt the operating conditions of the other unit operations to their use.

Obtaining p-hydroxycinnamic acids by extraction/purification from biomass seems to be the most relevant route in terms of development and exploitation, notably through the possible use of agro-industrial co-products. However, no flexible industrializable method applicable to all p-hydroxycinnamic acids is currently available.

The prior art reports studies of individual unit operations, such as the extraction of p-hydroxycinnamic acid derivatives (sinapine, chlorogenic acid), enzyme conversion into p-hydroxycinnamic acids or purification thereof. Only Thiel et al. (2015) have proposed an integrated separation method to simultaneously recover proteins, sinapic acid and phytic acid from rapeseed (WO 2015/181203 A1). This patent uses aqueous extraction with simultaneous alkaline hydrolysis followed by purification by adsorption on zeolites.

A method for obtaining polyphenol-enriched honey from plant material is disclosed in patent RO133390A0. The method described in this document comprises 7 steps. Of these 7 steps, 5 steps concern extraction (extraction and re-extraction), there is one honey enrichment step and finally one honey recovery step. One of the steps according to the method notably comprises the addition of an enzyme mixture consisting of hydrolases acting on cell wall polysaccharides and feruloyl esterases, followed by enzyme-assisted extraction of the plant material in water for 5 hours at 55-60° C. and pH 6.0.

Enzymatic conversion to obtain p-hydroxycinnamic acids following aqueous extraction was studied by Odinot et al. (2017). They have described a method for preparing a vinyl phenolic compound from a precursor hydroxycinnamic acid derived from an oilseed cake (WO2017/072450 A1). The enzymes used were produced by strains of Aspergillus Niger.

Creating industrial chains for the production of these p-hydroxycinnamic acids and their numerous high value-added derivatives requires access to these acids in relatively large volumes, but above all at sufficiently low cost. Added to these constraints is consumer interest in “natural” molecules as opposed to those derived from chemical synthesis.

There is a need for an industrializable process for purification of p-hydroxycinnamic acids from biomass.

### BRIEF SUMMARY

An innovative method for producing p-hydroxycinnamic acids in an aqueous medium by extraction and purification from lignocellulosic plant biomass has been developed.

More particularly, the method according to the present disclosure comprises the following two steps:
Step 1: Solid/liquid extraction with enzymatic hydrolysis at a pH below 5.5 of the p-hydroxycinnamic acids, which are either grafted to hemicelluloses or present in the form of ester derivatives; and
Step 2: Purification of the p-hydroxycinnamic acids released in step 1 by liquid/liquid extraction coupled to a membrane contactor composed of hollow fiber membranes from an aqueous medium into an organic solvent.

It particularly enables the industrial production of sinapic acid, ferulic acid and caffeic acid.

The method for producing p-hydroxycinnamic acids comprises the integration of several unit operations in order to reduce the number of steps and preserves the naturalness of the molecules by implementing eco-extraction processes using enzymes. More generally, this is a method for the selective purification of the p-hydroxycinnamic acids. The advantages of this method are numerous.

First, the method integrates extraction and enzymatic hydrolysis in a single step, without changing the medium. Thus, the two operations were optimized under constraints. The advantage of this integration is the reduction in volumes used, and the elimination of intermediate steps to change the solvent between extraction and conversion.

Furthermore, enzymatic hydrolysis of the p-hydroxycinnamic acids is achieved without any pH adjustment (no addition of base or acid). In fact, it has been shown that it is possible to use enzymatic cocktails directly at the pH of the biomass used, that is, at a pH lower than the pH of optimal conventional use. Indeed, hydrolysis takes place at a pH below 5.5 (and down to 4.2), whereas the p-hydroxycinnamoyl esterase activity of the enzyme mixtures is optimal at pH 6. At this pH of less than 5.5, the p-hydroxycinnamoyl esterase activity of the enzymatic cocktails used is sufficient for significant hydrolysis of the p-hydroxycinnamic acids (greater than or equal to 58% depending on the biomass used). This has two advantages: it eliminates the need to adjust the pH by adding acid solutions, and it therefore preserves the naturalness of the p-hydroxycinnamic acids.

Second, the proposed production method is green, sustainable and cost-effective. No organic solvents are used for solid/liquid extraction, and no salts are generated by chemical conversion. The molecules of interest produced by this method are therefore free of contaminants.

In particular, the innovative method combines enzymatic conversion with the use of a membrane contactor. During the enzymatic extraction-hydrolysis stage, the molecules are released into an aqueous medium. However, these molecules are hydrophobic in nature and will therefore tend to pass into the organic solvent at the contactor. The use of the membrane contactor therefore enables enrichment of the organic phase with the molecules of interest. This contributes to a high purification yield. The molecules of interest are then recovered in solid form by various conventional methods (evaporation, back-extraction). The use of a membrane contactor enables liquid/liquid extraction with a constant exchange surface and avoids the formation of emulsions when the aqueous and organic phases come into contact.

Third, the purification step is highly selective, reducing the number of unit operations required to recover the p-hydroxycinnamic acids with a high purity, and thus lowering the cost of the purification method.

Finally, thanks to the optimizations described above, the method can be scaled up for industrial production. The method therefore makes it possible to produce large volumes of p-hydroxycinnamic acids, moreover in an aqueous medium, opening the way to new applications in fields where the presence of solvents is forbidden, but also to any application requiring large quantities of p-hydroxycinnamic acids.

### BRIEF DESCRIPTION OF THE DRAWINGS

FIG. 1: Synthetic representation of the two steps of the method for producing p-hydroxycinnamic acids according to the present disclosure.

FIG. 2: Industrial representation of the method for producing p-hydroxycinnamic acids according to the present disclosure.

### DETAILED DESCRIPTION

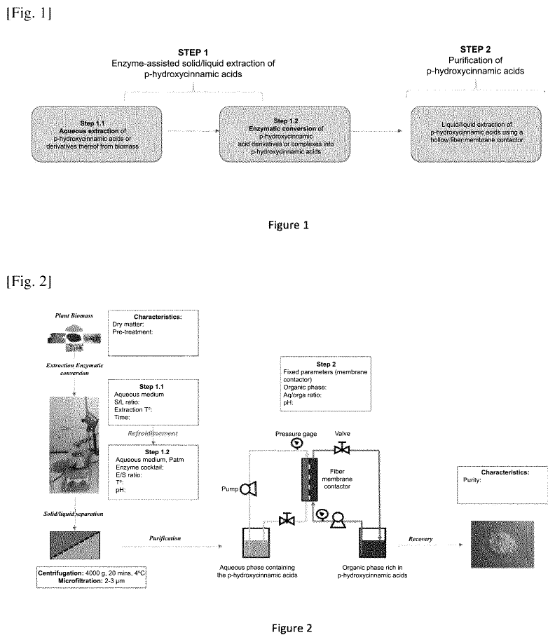

The present disclosure relates to a method for producing p-hydroxycinnamic acids from a lignocellulosic plant biomass comprising two steps, shown in FIG. 1:
Step 1: Solid/liquid extraction with enzymatic hydrolysis at a pH below 5.5 of the p-hydroxycinnamic acids, which are either grafted to hemicelluloses or present in the form of ester derivatives; and
Step 2: Purification of the p-hydroxycinnamic acids released in step 1 by liquid/liquid extraction coupled to a membrane contactor composed of hollow fiber membranes from an aqueous medium into an organic solvent.

The lignocellulosic plant biomass used in this method is advantageously a co-product of a conversion process such as pressing or milling, etc.

The p-hydroxycinnamic acids present in the biomass are released by enzymatic hydrolysis at acid pH. The pH depends on the nature of the biomass but is less than or equal to 5.5. It is obtained naturally without the addition of acid or basic solutions. Preferably, the pH is between 4 and 5.5.

The choice of an enzyme, or more generally an enzyme cocktail, depends on the nature of the biomass and the form wherein the p-hydroxycinnamic acids are found. This enzymatic cocktail will contain at least one p-hydroxycinnamoyl esterase, for example, a feruloyl esterase.

When the p-hydroxycinnamic acids are grafted onto hemicelluloses, hydrolysis is carried out by an enzymatic preparation (enzyme cocktail) comprising at least one p-hydroxycinnamoyl esterase and optionally another enzyme selected from a pectinase, a hemicellulase, a beta-glucanase, a xylanase, an arabinoxylase, etc.; the preparation adapted to the biomass is selected by the person skilled in the art.

Examples of suitable enzymatic cocktails are the following commercial mixtures: Pectinex Ultra SP-L, Celluclast 1.5L, Ultraflo L, Pectinase Protease MSD, D740L, D686L, D793L, D692L, DO40L, PO62L, C013L and G015L.

In a particular embodiment of the present disclosure, it is an enzyme cocktail. Examples of such cocktails allowing the release of p-hydroxycinnamic acids grafted onto hemicelluloses are:
Pectinex Ultra SP-L (mixture of pectinases, hemicellulases and beta-glucanases);
Celluclast 1.5L;
UltraFlo L (mixture of beta-glucanase and arabinoxylanase);
Pectinase PL;
Protease MSD;
D740L (mixture comprising a ferulic acid esterase (FAE), cellulase (CEL), and xylanase);
D686L (mixture comprising a beta-glucanase);
D793L (mixture comprising a beta-glucanase and a cellulase);
D692L (mixture comprising a ferulic acid esterase (FAE) and a cellulase);
D040L (mixture comprising a cellulase, pectinases and a beta-glucosidase) P062L;
C013L (mixture comprising a cellulase); and
G015L.

When p-hydroxycinnamic acids are present in the form of ester derivatives, hydrolysis is carried out by at least one enzyme such as rumen FAE or an enzymatic cocktail comprising at least one p-hydroxycinnamoyl esterase activity selected from Depol 740L, Ultraflo XL, Deltazym VR AC-100, Pectinase PL Amano, Celluclast 1.5L, Pectinex Ultra SP-L.

In a particular embodiment of the present disclosure, it is an enzyme cocktail. Examples of such cocktails allowing the release of p-hydroxycinnamic acids present in ester form are:
Depol 740L,
Ultraflo XL,
Deltazym VR AC-100,
Pectinase PL “Amano,”
Celluclast 1.5L, and
Pectinex Ultra SP-L.

The purification step must take place in a medium with a pH between 4 and 6. Either the plant matrix naturally allows acidification of the medium and the pH is suitable for purification step 2, or the hydrolysis step has led to an increase in pH and the pH needs to be adjusted to between 4 and 6. If necessary, the pH can be adjusted by adding an acid solution, with the person skilled in the art being familiar with conventional pH adjustment methods.

Step 2 is carried out in an aqueous medium using an organic solvent. Any organic solvent suited to the nature of the molecule of interest to be purified can be used.

The organic solvent, or more generally the mixture of organic solvents, to be used is selected from at least one of the following solvents: 4-methylpentan-2-one (MIBK), methoxycyclopentane (CPME), fatty alcohols (octanol, decanol, oleyl alcohol), fatty esters (octyl acetate, lauryl acetate).

The membrane contactor is a hollow fiber membrane contactor, but other configurations such as flat membranes could be used. Hollow fiber membrane contactors can vary in fiber number, length and diameter, porosity and number of pores, as well as the exchange surface.

The various stages of the selective purification process for p-hydroxycinnamic acids can be summarized as follows:
Having a lignocellulosic plant biomass;
Suspending in an aqueous solution after grinding, depending on the nature of the biomass;
Heating the aqueous solution (typically between 3° and 120° C. depending on the biomass);
Cooling to 30-50° C. (before adding enzymes);
Adding an enzymatic solution having a p-hydroxycinnamoyl esterase activity allowing either the release of acids grafted onto hemicelluloses, or the conversion of acids into ester derivatives;
Separating the solid and liquid extract by centrifugation;
Microfiltering (to remove particles having a diameter greater than 2-3 μm) to obtain an aqueous filtrate;
Adjusting the pH of the aqueous filtrate to between 4 and 6, if necessary; and
Purifying the acids by liquid/liquid extraction through a membrane with an organic phase.

The organic solvent used in step 2 can be selected from at least one of the following solvents: 4-methylpentan-2-one (MIBK), methoxycyclopentane (CPME), fatty alcohols (octanol, decanol, oleyl alcohol), fatty esters (octyl acetate, lauryl acetate). Preferably, the solvent is selected from MIBK or CPME, most preferably it is CPME.

This method therefore enables the purification of p-hydroxycinnamic acids from a lignocellulosic biomass and the recovery thereof in the organic phase. To have p-hydroxycinnamic acids in the aqueous phase, it is possible to carry out an additional back-extraction step from the organic phase.

This back-extraction step involves extracting p-hydroxycinnamic acid from the organic solvent either by evaporation for volatile solvents, or by liquid-liquid extraction for non-volatile solvents using a basic solution. The basic solution used for this back-extraction step can be, for example, a concentrated sodium hydroxide solution at 2.5 grams per liter with a pH of 12, but can also be of a different ionic nature, with the person skilled in the art being familiar with conventional basic solutions for back-extracting a compound.

In a particular embodiment of the present disclosure, the method enables sinapic acid to be produced from mustard bran and/or rapeseed cake, or any other biomass containing sinapic acid in free or bound form.

In another particular embodiment of the present disclosure, the method enables ferulic acid to be produced from wheat bran and/or beet pulp and/or corn cob and/or rice bran, or any other biomass containing ferulic acid in free or bound form.

In a particular embodiment of the present disclosure, the method enables caffeic acid to be produced from chicory roots and/or coffee grounds, or any other biomass containing caffeic acid in free or bound form.

The p-hydroxycinnamic acids produced using the method according to the present disclosure can be used as finished products or platform molecules for the fine chemicals, cosmetics, specialty chemicals, plant protection, agri-food and biomaterials markets.

### EXPERIMENTAL PART

### Example 1: Description of the General Principles of the Method for Extracting p-Hydroxycinnamic Acids According to the Present Disclosure

The method according to the present disclosure is composed of unit operations that have been optimized according to the initial biomass of the acid to be produced in order to provide a high yield and a high degree of purity. However, the general implementation remains the same for all types of biomass.

Four examples of implementation of this method from different biomasses are described below:
Recovering sinapic acid from mustard bran (Example 2);
Recovering sinapic acid from rapeseed cake (Example 3);
Recovering ferulic acid from wheat bran (Example 4); and
Recovering caffeic acid from chicory roots (Example 5).

For each example, a descriptive diagram specifying the operating conditions for each unit operation is provided; the general steps are shown in FIG. 2.

The p-hydroxycinnamic acid content was determined by liquid chromatography using standard solutions. The yield of p-hydroxycinnamic acid is determined according to equation 1:

$$R ( \% ) = \frac{{C}_{AH}}{{C}_{HA control process}} \times 100 \qquad Equation 1$$

With C_{HA}, the p-hydroxycinnamic acid concentration (mg/g_{Dry Matter}) obtained either by a unit operation or by the semi-integrated method proposed herein and C_{HA} control process, the p-hydroxycinnamic acid concentration (mg/g_{Dry Matter}) obtained by a control process.

The control process is made up of individually optimized unit operations, without studying the process as a whole. The control extraction was carried out in a mixture of 70% v ethanol and 30% v water at 75° C. The control conversion is based on chemical hydrolysis. It has been considered that the concentration obtained by the control process is the maximum concentration of p-hydroxycinnamic acid that can be obtained from the starting biomass.

The purity of the purified extract is determined according to equation 2:

$$P ( \% ) = \frac{{m}_{AH}}{{m}_{Purified dry matter}} \times 100 \qquad Equation 2$$

With ^{m}AH, the mass of p-hydroxycinnamic acid (mg) obtained either after each unit operation or after purification and ^{m}Purified dry matter, the mass of dry matter present in the purified extract.

The results obtained relate to the optimization of the enzyme-assisted extraction and purification method. Table 1 summarizes the operating conditions used for each of examples 2 to 5.

TABLE 1
Table 1: Operating conditions implemented as part of optimization

| | | Operating | | | | | | | |
|---|---|---|---|---|---|---|---|---|---|
| Step | | conditions | Example 2 | | Example 3 | | Example 4 | Example 5 | |
| Enzyme- | Extraction | S/L ratio | 10-100 | mL/g_{DM} | 100 | mL/g_{DM} | 2 to 10% DM | 1/10-1/50 | gM/mLS |

| | | | | | | | | | |
|---|---|---|---|---|---|---|---|---|---|
| assisted | in aqueous | Extraction | 30 to 100° | C. | 100° | C. | 30 to 60° | C. | T° amb-100° C. |

| | | |
|---|---|---|
| extraction | media | T° |

| | | | | | | | |
|---|---|---|---|---|---|---|---|
| Time | From 20 min | 40 | min | 6-24 | hours | 5-120 | mins |

to 2 hours 40
mins

| | | | | | |
|---|---|---|---|---|---|
| Hydrolysis | Enzymes | Depol 740L, | Deltazym VR | Ultraflo XL | Pectinase |
| | | Ultraflo XL, | AC-100 | (G)/Pectinase | PL |

| | Deltazym VR | | | | PL | | “Amano”/Pectinex |
|---|---|---|---|---|---|---|---|
| | AC-100, | | | | (P)/Celluclast | | Ultra SP/ |
| | Pectinase PL | | | | 1.5 L (C) | | |
| | Amano, | | | | | | |
| | Rumen FAE | | | | | | |
| T° | 30-75° | C. | 50° | C. | 30 to 60° | C. | T° amb-60° C. |

| pH | 4.2 ± 0.1 | | 5.5 | 3.75-5.75 | 4-to-8 |
|---|---|---|---|---|---|
| Enzyme | 60-1250 | μL/gDM | 1250 μL | G/C ratio: | 50 to 150 mg of |

| | Concentration | | (enzymatic | 20% to 100% | enzymatic |
|---|---|---|---|---|---|
| | | | solution)/gDM | From 0 to 2 | solution/gDM |
| | | | | mL per 100 | |
| | | | | mL (P) | |
| Purification | Solvents | MIBK, | MIBK, octyl | Ethyl acetate Octanol Decanol | |
| | (organic phase) | CPME, | acetate, oleyl | Oleyl alcohol MIBK | |
| | | octanol, octyl | alcohol | | |
| | | acetate, lauryl | | | |
| | | acetate | | | |
| | pH aqueous | 4.2 ± 0.1 | pH 4.0-5.9 | 5.5 | 5.5 |
| | medium | | | | |

| | | | |
|---|---|---|---|
| Aqueous/organic | (1:1) and | (1:1)v max. with | (1:1) and (1:2)v with plant |
| ratio | (1:2)v with | plant hydrolysate | hydrolysate (1:2)v-(1:5)v with |

| plant | model solution |
|---|---|
| hydrolysate | |
| (1:2)v- | |
| (1:10)v with | |
| model | |
| solution | |

| | |
|---|---|
| T° | Ambient |

Enzyme cocktails were screened by measuring their secondary activity (caffeoyl, feruloyl or sinapoyl esterase). To do this, the esterase activity of the cocktail was measured by spectrophotometry (difference in absorbance between the ester and acid forms of the molecules measured) on a model solution of p-hydroxycinnamic acid ester. The rate of ester conversion to p-hydroxycinnamic acid was measured over 10 minutes at temperatures of 25-35-45-50-55° C. and at a pH of 6-7-8. The cocktail selected is the one with the best conversion rate.

These screening experiments confirmed that enzymatic hydrolysis can be carried out at acidic pH, without pH adjustment and at the pH of the biomass, that is, up to a pH of between 4 and 5.5.

### Example 2: Production of Sinapic Acid from Mustard Bran

The mustard bran is suspended in an aqueous solution (milli-Q water). The mixture is stirred (mechanical stirring at 60 rpm) and heated to 100° C. for 20 minutes. There is a 30-minute rest period in a thermostatically-controlled oil bath at 40° C. Once the temperature has reached 40° C., the enzymatic solution is then added, enabling conversion to sinapic acid. Hydrolysis was carried out under optimal temperatures at a temperature of 40° C. and a pH of 4.2 for a conversion rate of sinapine to sinapic acid of 58%.

Solid-liquid separation is performed by centrifugation (4000 g, 20 minutes, 4° C.). The latter is filtered by microfiltration to remove particles having a diameter greater than 2-3 μm. The filtrate obtained corresponds to the aqueous phase. Purification is carried out by liquid/liquid extraction through a membrane (membrane contactor). An aqueous phase containing sinapic acid is brought into contact with an organic phase via a membrane. As sinapic acid has a greater affinity with the organic phase than with the aqueous phase, it will diffuse and enrich the organic phase. Depending on the type of organic phase, different methods can be used to recover sinapic acid in solid form. The yield of sinapic acid extraction was over 87%, with a purity of 49% when using MIBK as solvent and 62% when using CPME as solvent.

### Example 3: Production of Sinapic Acid from Rapeseed Cake

The rapeseed cake is suspended in an aqueous solution (milli-Q water). The mixture is stirred (mechanical stirring at 60 rpm) and heated to 100° C. for 20 minutes. Cooling is carried out in a thermostatically-controlled oil bath at 40° C. for 50 minutes to reach a temperature of 50° C. The enzymatic solution is then added, enabling conversion to sinapic acid. Hydrolysis was carried out under optimal temperatures at a temperature of 50° C. and a pH of 5.5 for a conversion rate of sinapine to sinapic acid greater than 80%.

Solid-liquid separation is performed by centrifugation (4000 g, 20 minutes, 4° C.). The latter is filtered by microfiltration to remove particles having a diameter greater than 2-3 μm. The filtrate obtained corresponds to the aqueous phase. This is acidified to pH 4.5 with acetic acid. Purification is carried out by liquid/liquid extraction through a membrane (membrane contactor). An aqueous phase containing sinapic acid is brought into contact with an organic phase via a membrane. As sinapic acid has a greater affinity with the organic phase than with the aqueous phase, it will diffuse and enrich the organic phase. Depending on the type of organic phase, different methods can be used to recover sinapic acid in solid form. The yield of sinapic acid extraction is over 81% when using MIBK or octyl acetate as solvent.

### Example 4: Ferulic Acid Production from Wheat Bran

Wheat bran (not starch-free) is ground and then sieved to obtain particle sizes ranging from 180 μm to 850 μm. The wheat bran is then autoclaved (121° C. for 20 minutes) and suspended in an aqueous solution (milli-Q water) at 50° C. The enzymes are then added to the medium, which continues to be stirred for 24 hours, enabling ferulic acid to be obtained from the lignocellulose in the wheat bran. Hydrolysis was carried out under optimal conditions at a temperature of 50° C. and a pH of 4.2 with a ferulic acid release rate of 71%.

Solid-liquid separation is performed by centrifugation (4000 g, 20 minutes, 4° C.). The latter is filtered by microfiltration to remove particles having a diameter greater than 2-3 μm. The filtrate obtained corresponds to the aqueous phase. Purification is carried out by liquid/liquid extraction through a membrane (membrane contactor). An aqueous phase containing ferulic acid is brought into contact with an organic phase via a membrane. As ferulic acid has a greater affinity with the organic phase than with the aqueous phase, it will diffuse and enrich the organic phase. Depending on the type of organic phase, different methods can be used to recover ferulic acid in solid form. The yield of ferulic acid extraction was over 85%, with a purity of 46% when using MIBK as solvent and 52% when using CPME as solvent.

### Example 5: Production of Caffeic Acid from Chicory Co-Products

Dried and ground chicory roots (<500 μm) are suspended in an aqueous solution (milli-Q water). The enzymatic solution is added to simultaneously extract chlorogenic acid and convert it into caffeic acid. The mixture is then stirred with a magnetic stirrer (600 rpm) and heated to 30° C. for 60 minutes. Hydrolysis was carried out under optimal conditions at a temperature of 30° C. and a pH of 4.2 for a conversion rate of chlorogenic acid to caffeic acid greater than 98%.

Solid-liquid separation is performed by centrifugation. The latter is filtered by microfiltration to remove particles having a diameter greater than 2-3 μm. The filtrate obtained corresponds to the aqueous phase. Purification is carried out by liquid/liquid extraction through a membrane (membrane contactor). An aqueous phase containing caffeic acid is brought into contact with an organic phase via a membrane. As caffeic acid has a greater affinity with the organic phase than with the aqueous phase, it will diffuse and enrich the organic phase. Depending on the type of organic phase, different methods can be used to recover caffeic acid in solid form.

1. A method for producing p-hydroxycinnamic acids from a lignocellulosic plant biomass, comprising the following two steps:
step 1: solid/liquid extraction with enzymatic hydrolysis carried out at a pH below 5.5 of the p-hydroxycinnamic acids, which are either grafted to hemicelluloses or present in the form of ester derivatives; and
step 2: purification of the p-hydroxycinnamic acids released in step 1 by liquid/liquid extraction coupled to a membrane contactor composed of hollow fiber membranes from an aqueous medium into an organic solvent.

2. The method according to claim 1, wherein the enzymatic hydrolysis is carried out using an enzymatic cocktail comprising at least one p-hydroxycinnamoyl esterase.

3. The method of claim 2, wherein the solid/liquid extraction with enzymatic hydrolysis is carried out at a pH between 4 and 5.5.

4. The method of claim 3, further comprising adjusting a pH of a medium resulting at the end of step 1 to between 4 and 6 in step 2.

5. The method of claim 4, wherein the organic solvent is selected from at least one of the following solvents: 4-methylpentan-2-one (MIBK), methoxycyclopentane (CPME), a fatty alcohol, or a fatty ester.

6. The method of claim 5, further comprising a step of back-extracting the p-hydroxycinnamic acids from the organic solvent, either by evaporation for volatile solvents, or by liquid-liquid extraction for non-volatile solvents, using a basic solution.

7. The method of claim 5, wherein the p-hydroxycinnamic acid is sinapic acid from mustard bran and/or rapeseed cake.

8. The method of claim 5, wherein the p-hydroxycinnamic acid is ferulic acid from wheat bran, beet pulp, and/or corn cob.

9. The method of claim 5, wherein the p-hydroxycinnamic acid is caffeic acid from chicory roots and/or coffee grounds.

10. The method of claim 1, wherein the solid/liquid extraction with enzymatic hydrolysis is carried out at a pH between 4 and 5.5.

11. The method of claim 1, further comprising adjusting a pH of a medium resulting at the end of step 1 to between 4 and 6 in step 2.

12. The method of claim 1, wherein the organic solvent is selected from at least one of the following solvents: 4-methylpentan-2-one (MIBK), methoxycyclopentane (CPME), a fatty alcohol, or a fatty ester.

13. The method of claim 1, further comprising a step of back-extracting the p-hydroxycinnamic acids from the organic solvent, either by evaporation for volatile solvents, or by liquid-liquid extraction for non-volatile solvents, using a basic solution.

14. The method of claim 1, wherein the p-hydroxycinnamic acid is sinapic acid from mustard bran and/or rapeseed cake.

15. The method of claim 1, wherein the p-hydroxycinnamic acid is ferulic acid from wheat bran, beet pulp, and/or corn cob.

16. The method of claim 1, wherein the p-hydroxycinnamic acid is caffeic acid from chicory roots and/or coffee grounds.
